(19) United States
(12) Patent Application Publication
(10) Pub. No.: US 2011/0307001 A1
(43) Pub. Date: Dec. 15, 2011

(54) OSCILLATING TISSUE DISSECTOR

(76) Inventor: Hilton BECKER, Boca Raton, FL (US)

(21) Appl. No.: 13/157,044

(22) Filed: Jun. 9, 2011

Related U.S. Application Data
(60) Provisional application No. 61/352,976, filed on Jun. 9, 2010.

(51) Int. Cl. A61M 29/00

(52) U.S. Cl. 606/192

(57) ABSTRACT

Provided is a tissue dissector that includes: a tube having a proximal end and a distal end; a rod disposed within the tube, ribs attached to the rod and extending outside of the tube; a housing disposed at the proximal end of the tube; an actuator disposed in the housing, to rotationally oscillate the rod with respect to the tube; and a first seal disposed around the rod, to prevent fluid from entering the tube.

### CROSS-REFERENCE TO RELATED APPLICATION

This application claims priority from and the benefit under 35 U.S.C. §119(a) of U.S. Provisional Patent Application No. 61/352,976, filed on Jun. 9, 2010, which is incorporated by reference for all purposes as if fully set forth herein.

### BACKGROUND OF THE INVENTION

1. Field of the Invention

Exemplary embodiments of the present invention relate to an oscillating tissue dissector.

2. Discussion of the Background

Liposuction refers to the surgical removal of adipose tissue from fat deposits by agitation and suction. In conventional liposuction, a cannula, which may be a stainless steel tube having one or more holes in its distal end, is inserted into an incision and positioned into adipose tissue, between the dermal layers and the underlying muscle tissue. Typically, a suction device is attached to the proximal end of the cannula, i.e., the end of the cannula held by the user.

A cannula is moved through adipose tissue in a back-and-forth manner, to create tunnels within the adipose tissue. This back-and-forth movement may be enhanced by using a vibration device, as the fat is suctioned by the suction device through the cannula, decreasing the volume of the adipose tissue. Between one and twelve, and sometimes more than twelve, incisions may be made during a liposuction procedure with the incisions being linear incisions or adits and are typically 3 mm to 20 mm in length or diameter, respectively. Ideally, incisions are as small as possible in attempt to make them less visible.

Liposuction may take multiple hours to perform with a conventional cannula, and multiple liposuction techniques are currently in use, including laser-assisted liposuction (LAL), power-assisted liposuction (PAL), ultrasonic assisted liposuction (UAL), and water jet assisted liposuction. PAL and UAL vibrate the cannula at sonic and ultrasonic frequencies, respectively. Moreover, LAL, PAL, and UAL deliver energy to the adipose tissue to be resected, while water jet assisted liposuction uses a high pressure, directed water jet to disturb the adipose tissue.

However, the heat generated by LAL, PAL, and UAL burns tissue, thereby resulting in scarring that causes the skin to adhere to the underlying muscle. In addition, such cannulae may cause damage to surrounding tissue, blood vessels, and nerves. Furthermore, since the size of such cannulae is increased to encompass both a suction tube and a tissue dissecting apparatus, the above problems may be exacerbated.

In particular, if the diameter of a cannula is enlarged, e.g., in a dissecting cannula where outward projections facilitate fat breakdown, a larger incision is generally required. Trauma to the skin during UAL procedures, due to local heating caused by the ultrasonic vibrations can cause damage. Further, many conventional cannulae may cause bruising, damage to connective tissue, discomfort, pain, scarring, swelling, and tenderness. Additionally, the motion of conventional cannulae through the adipose tissue may require not only manual dexterity but also physical stamina, due to the length of the operation and the force that must be applied to guide the cannula within tissue.

Recently, liposuction techniques have been developed wherein some dissected tissue is allowed to remain in the body. In particular, dissected tissue is moved to other areas of the body where it serves as tissue scaffolding as it is absorbed by the body. Thus, there is a need for a tissue dissector that does not include a cannula.

An efficient dissector that reduces liposuction operating duration and a user's physical exertion are desired. Decreasing the deleterious effects of typical cannulae involved in the production of bruising, swelling, and nerve and tissue damage necessitate the development of a next generation of cannulae.

### SUMMARY OF THE INVENTION

Exemplary embodiments of the present invention provide for a tissue dissector that may more efficiently dissect adipose tissue with less damage to surrounding tissue, blood vessels, and nerves, while being able to be inserted into a relatively small surgical incision.

An exemplary embodiment of the present invention provides a tissue dissector that includes: a tube having a proximal end and a distal end; a rod disposed within the tube, ribs attached to the rod and extending outside of the tube; a housing disposed at the proximal end of the tube; an actuator disposed in the housing, to rotationally oscillate the rod with respect to the tube; and a first seal disposed around the rod, to prevent fluid from entering the tube.

Once the fatty tissues have been sufficiently broken down, a standard suction cannula may be used to aspirate some or all of the fatty tissue. Any remaining fatty tissue remains viable, as it has not been subjected to laser irradiation or ultrasound. The remaining tissue can be sculpted into a desired shape.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and, together with the description, serve to explain the principles of the invention.

FIG. 1 illustrates a sectional view of a tissue dissector, according to an exemplary embodiment of the present invention.

FIGS. 2A, 2B, 2C, and 2D illustrate top and side views of ribs of a tissue dissector, according to exemplary embodiments of the present invention.

FIG. 3A illustrates sectional view of a tissue dissector, according to an exemplary embodiment of the present invention.

FIG. 3B illustrates a perspective view of a tube of the tissue dissector of FIG. 3A.

FIGS. 4A, 4B, 4C, and 4D illustrate the operation of an actuator of the tissue dissector of FIG. 2

### DETAILED DESCRIPTION OF THE ILLUSTRATED EMBODIMENTS

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of elements and regions may be exaggerated for clarity. Like reference numerals in the drawings denote like elements.

It will be understood that when an element is referred to as being “on,” “coupled to,” or “connected to” another element, it can be directly on, directly coupled to, or directly connected to the other element, or intervening elements may be present. In contrast, when an element is referred to as being “directly on,” “directly coupled to,” or “directly connected to” another element, there are no intervening elements present.

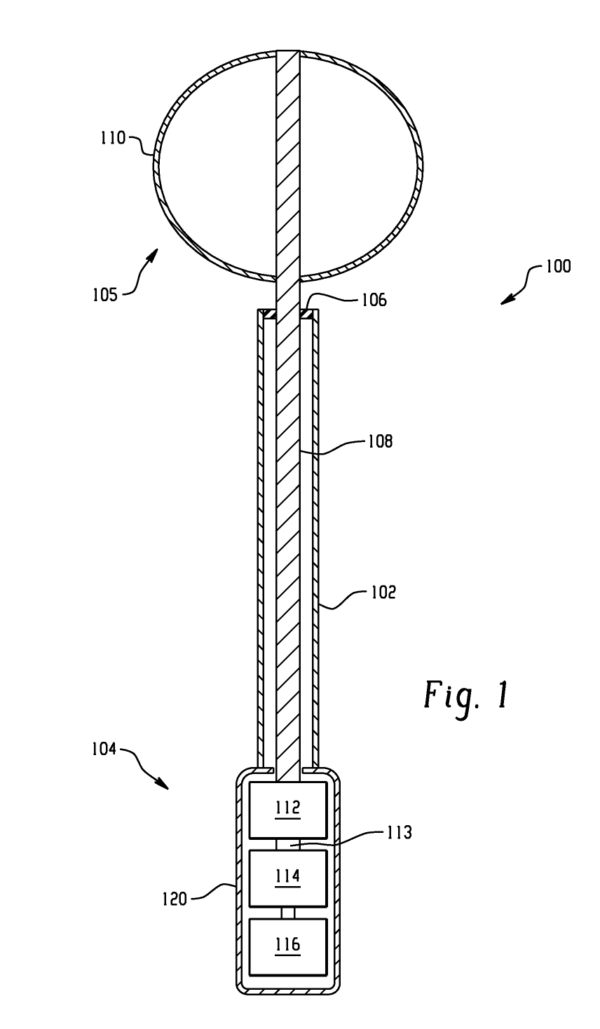

FIG. 1 is a cross-sectional view showing a tissue dissector 100, according to a first exemplary embodiment of the present invention. Referring to FIG. 1, the tissue dissector 100 has a proximal end 104 and a distal end 105. The tissue dissector 100 includes a tube 102, a rod 108 that extends through the tube 102, and ribs 110. The ribs 110 are connected to the distal end the rod 108, which is exposed outside of the tube 102.

The ribs 110 may be formed of a metal, such as stainless steel. The ribs 110 may be attached to the rod 108 in any suitable manner. For example, the ribs 110 may be welded to the rod 108. While two ribs 110 are shown in FIG. 1, the present invention is not limited to any particular number of ribs. For example, from two to eight ribs may be included. The ribs 110 may be circular in cross-section, i.e., wire-shaped. However, the ribs 110 may also be rectangular in cross-section, i.e., ribbon-shaped, ovoid in cross-section, or triangular in cross-section. However, the present invention is not limited to any particular type of rib shape.

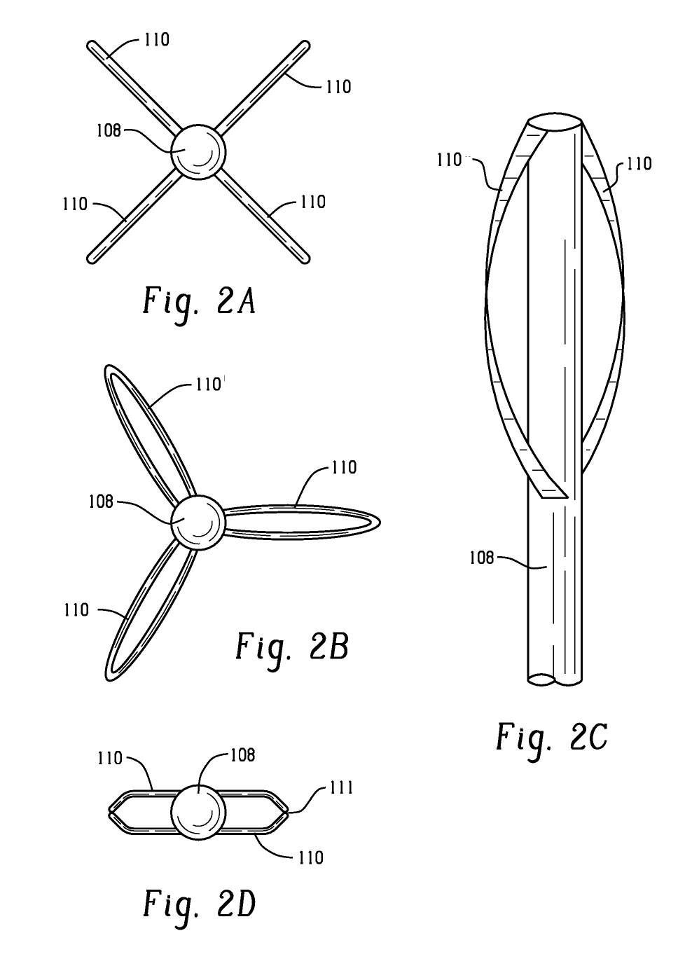

FIGS. 2A, 2B, 2C and 2D illustrate top and perspective views of rods 108 having various numbers of the ribs 110 attached thereto, in various configurations. Referring to FIG. 2A, instead of the two ribs 110 as shown in the embodiment of FIG. 1, four ribs 110 are attached to the rod 108. The ribs 110 are attached to the rod 108 such that the ribs 110 have a single radius of curvature. In other words, the ribs 110 are attached to points on the rod 108 that are disposed on a line extending along the length of the rod 108.

Referring to FIG. 2B, three ribs 110 are attached to the rod 108. However, the ribs 110 are attached to the rod 108 such that the ribs 110 have two radii of curvature. In other words, the attachment points of the ribs 110 to the rod 108 are not disposed on a line extending along the length of the rod 108.

Referring to FIG. 2C, two ribs 110 are attached to the rod 108. The ribs 110 are ribbon-shaped, in contrast to the wire-shaped rods 110 of FIGS. 1, 2A, and 2B. The ribs 110 of FIG. 2C are attached to the rod 108, such that the ribs 110 have two radii of curvature.

Referring to FIG. 2D, four ribs 110 are attached to the rod 108. The ribs 110 are attached to the rod 108 in positions similar to those shown in FIG. 2A. However, adjacent ones of the ribs 110 are also bent so as to contact one another at contact points 111, thereby creating second radii of curvature. Due to the contact points 111, the rigidity of the ribs 110 is increased.

As recited above, various numbers of ribs 110 may be attached to the rod 108, and the ribs may have more than one radius of curvature. Additionally, a combination of fused and un-fused ribs 110 may be used. However, as a general principal, the ribs 110 should be symmetrically attached to the rod 108, such that the resultant structures are balanced when oscillated, as discussed below.

Referring again to FIG. 1, the tube 102 may be formed of a rigid material, such as stainless steel or the like. The tube 102 may be from about 200 to about 400 mm long and from about 3 to about 5 mm in diameter. For example, the tube 102 may be 300 mm long and may have a diameter of about 4 mm. As such, the tube 102 is substantially thinner than a conventional liposuction cannula, since the tube 102 is not used to remove adipose tissue.

The rod 108 may be formed of a rigid material or a flexible material having a high torsional rigidity. For example, the rod 108 may be made of a metal, plastic, or a composite material. According to some embodiments, the rod 108 is formed of stainless steel.

A seal 106 is disposed around the rod 108 at the distal end of the tube 102. The seal 106 prevents fluid from entering the tube 102. The seal 106 may be, for example, a rubber or silicon gasket. According to some embodiments, the seal 106 may include a bearing that prevents motions of the rod 108 from being transferred to the tube 102.

A housing 120 is connected to the proximal end of the tube 102. The housing 120 may be shaped to operate as a handle. A transmission 112, an actuator 114, and a controller 116 are disposed in the housing 120. The actuator 114 oscillates the rod 107 via the transmission 112 and under the control of the controller 116. In particular, the actuator 114 is connected to the transmission 112 by a drive shaft 113. The actuator 114 rotates the drive shaft 113, and the transmission 112 converts the rotational motion of the drive shaft 113 into an oscillating motion. As referred to herein, oscillation refers to a non-rectilinear (rotational) reciprocation.

In particular, the transmission may rotate the rod 108 clockwise and counter clockwise in, for example, a 30 degree arc. However, the present invention is not limited thereto, as the rod 108 may be oscillated in arcs of from 20 degrees to 40 degrees. The operation of the actuator 114 is controlled by the controller 116. The controller may be connected to a power source (not shown) or may include a power source. The power source may be an AC voltage source or a rechargeable battery. The controller may vary the amount of current supplied to the actuator 114, in order to vary the amount of torque produced by the actuator 114, as discussed below.

The actuator 114 may be an electric motor that operates using a DC voltage or an AC voltage, depending on the type of power supply. The controller 116 may include a central processing unit. If the actuator 114 operates using a DC voltage, the controller 115 may include a power rectifier to convert an AC voltage into a DC voltage.

During tissue dissection, the tissue dissector 100 is inserted into a patient, and the oscillating ribs 110 impact target tissues, thereby dissecting the tissues. The tissue may be adipose tissue and/or fibrous tissue, such as, lipomas, abnormal collections of fatty tissue e.g., Buffalo hump deformities, gynecomastia (abnormal male breast deformity) tissue, and the like.

If the ribs 110 are formed of a flexible material, the ribs 110 maybe bent towards the rod 108 when the ribs 110 encounter fibrous tissue. As a result, the torque applied by the ribs 110 to such tissue increases, in accordance with the amount of bending. As a result, the force applied to such tissue may be automatically increased, thereby allowing the tissue dissector 100 to breakup fibrous tissue. In addition, the ribs 110 can be bent during insertion into a patient, to decrease the size of a corresponding incision.

According to some embodiments, the controller 116 can detect the presence of fibrous tissue by, for example, monitoring the resistance encountered by the actuator 114. In such circumstances, the controller 116 can increase the amount of current applied to the actuator 114, to thereby increase the torque applied to the ribs 110. According to other embodiments, the torque applied to the ribs 110 can be manually controlled.

Since the tube 102 is isolated from the movement of the rod 108 by the seal/bearing 106, the tube 102 does not oscillate during tissue dissection. As such, friction between the tube 102 and non-target tissue, for example skin, is greatly decreased. Accordingly, friction burns and/or bruising can be substantially reduced. In addition, the tissue dissector 100 is easier and less strenuous to use, since the handle is also isolated from the movement of the rod 108.

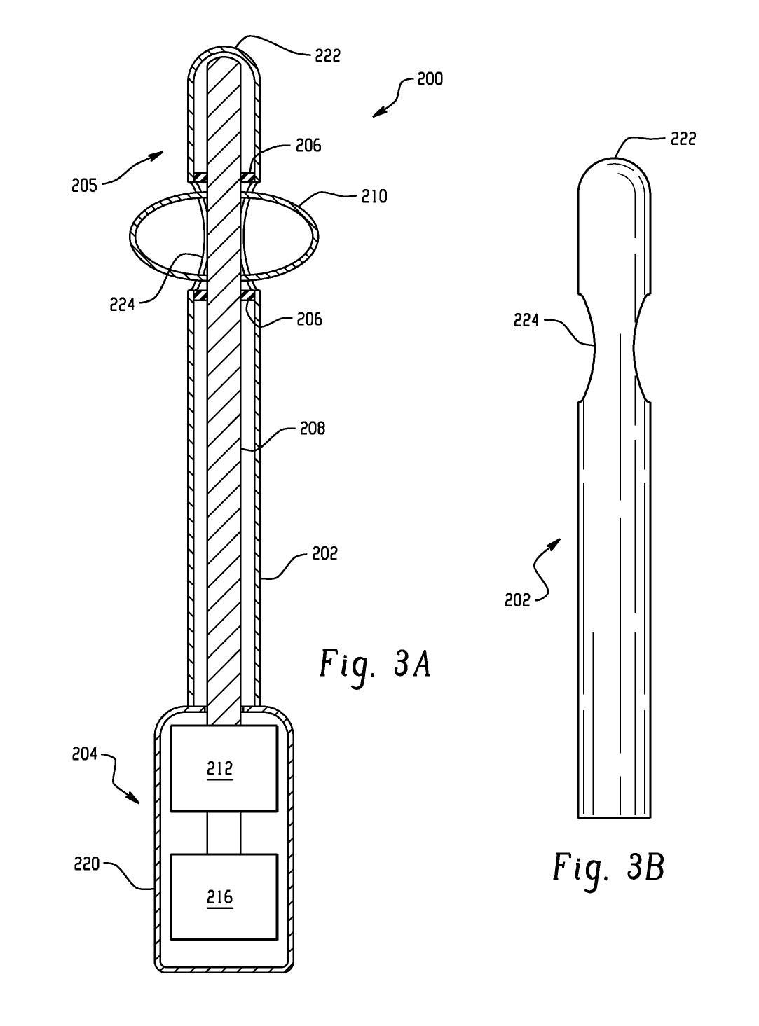

FIG. 3 illustrates a sectional view of a tissue dissector 200, according to another exemplary embodiment of the present disclosure. FIG. 3B is a perspective view of a tube 202 shown in FIG. 3. The tissue dissector 200 is similar to the tissue dissector 100 shown in FIG. 1, thus, only the differences therebetween will be described in detail.

Referring to FIGS. 3A and 3B, the tissue dissector 200 has a proximal end 204 and a distal end 205. The tissue dissector 200 includes the tube 202, a rod 208 disposed within the tube 202, ribs 210 connected to the rod 208, and a housing 120 connected to the proximal end of the tube 202. An actuator 212 and a controller 216 are disposed in the housing 220.

Unlike the tissue dissector 100, the ribs 210 are offset from the distal end 205 of the tissue dissector 200. In particular, the ribs 210 are offset from the distal end of the rod 208. The tube 202 includes a rounded distal end 222 and openings 224 disposed adjacent to the distal end 222. The rod 208 extends into the distal end 222, such that the ribs 210 extend out of the openings 224. The openings 224 are recessed, such that the ribs 210 can oscillate without contacting the tube 208.

While two openings 224 and two ribs 210 are shown in FIGS. 3A and 3B, the present invention is not limited thereto. In particular, other numbers of ribs 210 may be included, so long as the openings 224 are modified accordingly. For example, the openings can be shaped to each accommodate two of the ribs 210, such that the tissue dissector 220 includes a total of four of the ribs 210. In the alternative, three of the openings 224 can be provided, if the tissue dissector 220 includes three of the ribs 210.

The tissue dissector 200 includes seals 206 disposed between the rod 208 and the tube 202. The seals 206 prevent fluid from entering the tube 202. The seals 206 may be gaskets or bearings, as recited above. As such, the seals 206 isolate the tube from the motion of the rod 208.

The distal end of the tube 202 is generally rounded. However, the distal end may be various shapes such as flat, oval, and oblong and may include various terminal shapes, such as a tapered or a bifurcated, V-shape end. The tube 202 may also be round or various shapes such as flat, oval, and oblong. When in use, the distal end may move aside blood vessels and nerves. As such, the ribs 210, although breaking down the adipose tissue, may not harm other surrounding tissues such as dermal tissue or nerves, thereby reducing bruising, nerve damage, tissue damage, and pain.

The actuator 212 is connected to the rod 208 and oscillates the rod 208 under the control of the controller 216. The actuator 212 may be driven using a vacuum source (not shown) or a compressed air source (not shown).

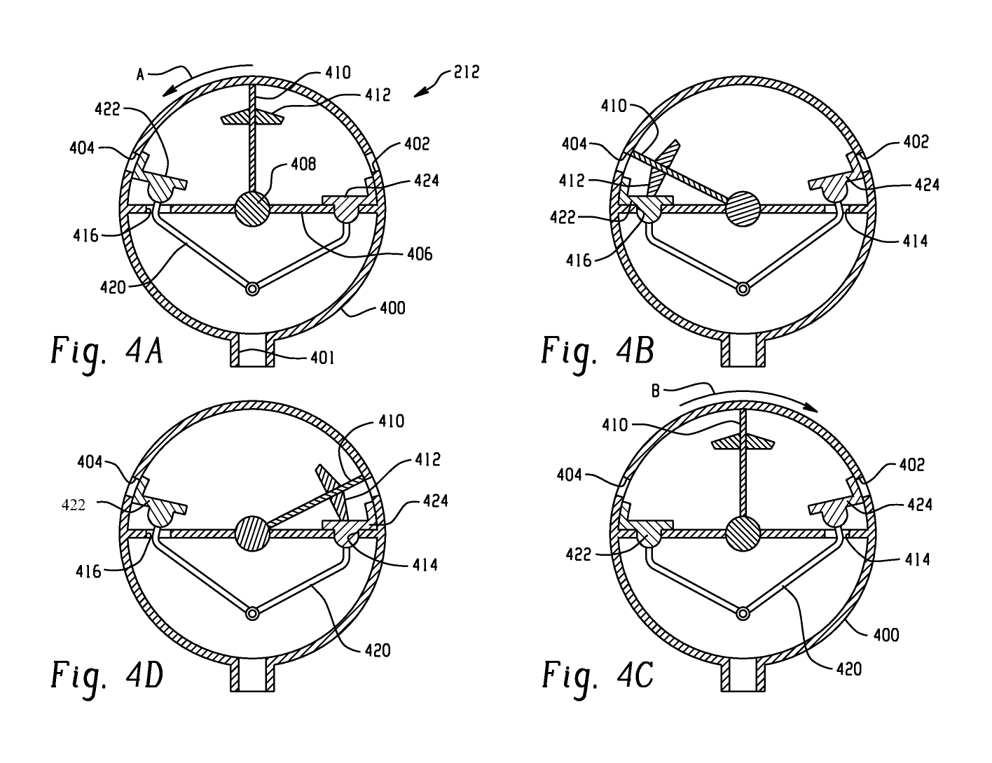

FIGS. 4A, 4B, 4C, and 4D illustrate sectional views of the actuator 212 while in operation. Referring to FIGS. 4A-4D, the actuator 212 includes a housing 400 having a connection port 401 and vents 402, 404 formed therein. The connection port 401 can be connected to a suction source (not shown). A baffle 406 is disposed inside the housing and divides the internal area of the housing 400 into first and second areas. The baffle 406 includes openings 414 and 416.

The actuator 212 also includes a rod 408, a vane 410 connected to the rod 408, gaskets 422, 424, and a linkage 420 connecting the gaskets 422, 424. The gaskets 422, 424 each include two protrusions that selectively seal the vents 402, 404 and the openings 414 and 416. The vane 410 includes protrusions 412 that contact the gaskets 422, 424. Movements of the vane 410 rotate the rod 408, which is attached to the rod 208.

Referring to FIG. 4A, suction is applied to the connection port 401. Due to the positions of the gaskets 422, 242, the aperture 414 and vent 404 are sealed, and the vent 402 and aperture 416 are open. Accordingly, the suction is applied to a first side of the vane 410 through the aperture 416, such that the vane 410 is moved in direction A. As the vane 410 is moved in direction A, external air is drawn into the housing 400 through the vent 402.

Referring to FIG. 4B, one of the protrusions 412 contacts the gasket 422, such that the gasket 422 moves toward the baffle 406. As such, the gasket 422 seals the aperture 416 and opens the vent 404. The movement of the gasket 422 is transferred to the gasket 424 by the linkage 420. As a result, the gasket 424 is moved away from the baffle 406, thereby sealing vent 402 and opening the aperture 414.

As shown in FIG. 4C, the suction is then applied to a second side of the vane 410, thereby moving the vane 410 in direction B. As the vane 410 is moved in direction B, external air is drawn into the housing 400 through the vent 404 by the movement of the vane 410.

Referring to FIG. 4D, one of the protrusions 412 contacts the gasket 424, such that the gasket 424 moves toward the baffle 406, and gasket 422 moves away from the baffle 406. As a result, the suction is again applied to the first side of the vane 410, and the vane 410 begins to move in direction A, and the above process is repeated.

Thus, the vane 410 oscillates the rod 408, which oscillates the rod 208. Accordingly, the tissue dissector 200 can be driven using a suction source commonly found in an operating room. In addition, once a tissue has been dissected, the suction source can be attached to a conventional cannula for liposuction of the dissected tissue.

Further, the degree of oscillation can be varied, by varying the location of the baffle 406 and/or by angling opposing sides of the baffle 406 with respect to the rod 408. According to some aspects, the protrusions 412 may be omitted, and the vane 412 may directly contact the gaskets 422, 424. According to other aspects, the actuator 212 can be driven using compressed air.

The tissue dissector 200 may be operated in a manner similar to that of the tissue dissector 100. In addition, the actuator 212 can be substituted for the actuator 114 and transmission 112, and vice versa. According to some aspects, the overall lengths of the tissue dissectors 100, 200 may range from about 10 cm to 80 cm, and the length of the tubes 102, 202 may range from 9 cm to 79 cm. The outer diameters of the tubes 102, 202 may range from several millimeters to several centimeters.

With respect to the ribs 110, 210, the total length of the ribs 110, 210 may range from 1 cm to 8 cm. The ribs 110, 210 a radius of curvature ranging from 170 to 190 degrees. The surface of the ribs 110, 210 may be generally smooth and/or rounded.

The dissection of adipose tissue may be highly efficient using the tissue dissectors 100, 200, as compared with conventional tissue dissectors, due to a number of factors. First, the larger cross section of the ribs creates a larger cross sectional area of tunnels formed within the adipose tissue. Further, besides the increased cross sectional area of the tunnels, the ribs may contact more adipose tissue per thrust movement by virtue of the shape of the ribs.

Since the tube of the tissue dissector is not oscillated, unwanted skin irritation and burning are reduced, and the tissue dissector is less strenuous to operate. In addition, since the tube is thinner than conventional liposuction cannula, the tissue dissector can be inserted into a smaller incision.

It will be apparent to those skilled in the art that various modifications and variations can be made in the present invention without departing from the spirit or scope of the invention. Thus, it is intended that the present invention covers the modifications and variations of this invention provided they come within the scope of the appended claims and their equivalents.

What is claimed is:

1. A tissue dissector comprising:
a tube having a proximal end and a distal end;
a rod disposed within the tube;
ribs attached to the rod and extending outside of the tube;
a housing disposed at the proximal end of the tube;
an actuator disposed in the housing, to rotationally oscillate the rod with respect to the tube; and
a first seal disposed around the rod, to prevent fluid from entering the tube.

2. The tissue dissector of claim 1, wherein:
a portion of the rod is exposed outside of the distal end of the tube; and
the ribs are attached to the exposed portion of the rod.

3. The tissue dissector of claim 2, wherein the ribs comprise:
a first rib disposed on a first surface of the rod; and
a second rib disposed on an opposing second surface of the rod.

4. The tissue dissector of claim 2, wherein the ribs comprise a first rib, a second rib, and a third rib that are disposed equidistantly around the rod.

5. The tissue dissector of claim 2, wherein the ribs have one radius of curvature or two radii of curvature.

6. The tissue dissector of claim 2, wherein the tissue dissector comprises four of the ribs.

7. The tissue dissector of claim 2, wherein at least two of the ribs are fused together.

8. The tissue dissector of claim 1, wherein the first seal comprises a bearing to isolate the tube from the oscillations of the rod.

9. The tissue dissector of claim 1, further comprising a transmission disposed in the housing, wherein:
the actuator is an electric motor; and
the transmission is disposed between the motor and the rod, to convert a rotational motion of the motor into the rotational oscillation of the rod.

10. The tissue dissector of claim 1, wherein the rod has a diameter of between about 2 mm and about 10 mm.

11. The tissue dissector of claim 1, further comprising a controller to control the actuator.

12. The tissue dissector of claim 1, wherein the actuator is powered using a vacuum source or compressed air.

13. The tissue dissector of claim 1, wherein:
the distal end of the tube is closed;
the tube comprises openings disposed adjacent to the distal end; and
the ribs extend out of the openings.

14. The tissue dissector of claim 13, wherein:
the tube comprises two of the openings and two of the ribs; and
each rib extends out of a respective one of the openings.

15. The tissue dissector of claim 13, further comprising a second seal disposed around the rod, wherein:
the first seal is disposed between the ribs and the distal end of the tube; and
the second seal is disposed between the ribs and the proximal end of the tube.

16. The tissue dissector of claim 15, wherein the seals comprise bearings to isolate the tube from the oscillations of the rod.

17. The tissue dissector of claim 13, wherein the distal end of the tube is rounded.

18. The tissue dissector of claim 1, wherein the rod oscillates the ribs in arcs of from 20 degrees to 180 degrees.

19. The tissue dissector of claim 1, wherein the actuator comprises:
a housing comprising a first vent and a second vent;
a rod disposed within the housing;
a baffle to divide the interior of the housing into a first area and a second area, the baffle comprising a first aperture and a second aperture;
a first gasket to selectively close the first vent or the first aperture;
a second gasket to selectively close the second vent or the second aperture; and
a vane to oscillate the rod, wherein,
when suction is applied to the first area and the second aperture is sealed by the second gasket, the suction is applied to a first side of the vane via the first aperture, to move the vane in a first direction, and
when suction is applied to the first area and the first aperture is sealed by the first gasket, the suction is applied to a second side of the vane via the second aperture, to move the vane in a second direction.

20. The tissue dissector of claim 19, wherein the actuator further comprises:
a linkage connecting the first and second gaskets, such that when one of the gaskets is moved towards the baffle, the other gasket is moved away form the baffle.
